(19) United States
(12) Patent Application Publication
(10) Pub. No.: US 2007/0203478 A1
(43) Pub. Date: Aug. 30, 2007

(54) Method and system for elasto-modulation of ocular tissue

(76) Inventor: Satish V. Herekar, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/706,803

(22) Filed: Feb. 14, 2007

Related U.S. Application Data
(60) Provisional application No. 60/775,650, filed on Feb. 21, 2006.

(51) Int. Cl. A61B 18/18; A61H 1/00; A61H 5/00; A61H 1/02

(52) U.S. Cl. 606/10; 606/4; 601/2; 601/15

(57) ABSTRACT

Excitation and radiation techniques and apparatus for performing customized elasto-modulation of ocular tissue in an annular region around the cornea, such as for the purpose of ciliary translocation (ACT) or promotion of aqueous outflow to relieve ocular pressure are provided utilizing a transconjunctival incisionless scleral treatment. Laser radiation is for example applied to scleral tissue to decrease zonular slack, or alter equatorial offsets between lens and ciliary muscles as a treatment for presbyopia by way of example.

### BACKGROUND OF THE INVENTION

The present invention relates generally to biomedical techniques. More particularly, the invention relates to a method and apparatus for performing customized ciliary translocation, such as in the anterior region of the ciliary muscle and outward therefrom.

Presbyopia is a loss or reduction of near focus due to the decreased accommodating power of the eye, which generally takes place as people age. Various presbyopia correction surgical techniques have been proposed/attempted, such as scleral expansion segments (e.g., scleral expansion via bands (SEB)), scleral implants(bulbous), scleral perforations(LaserAce), annular inserts(capsular/zonular), anterior ciliary sclerostomy/limbal relaxing incisions (ACS/LRI) with/without inserts, scleral ablation (SA), intraocular(epithelial, stromal, anterior chamber etc) implant techniques such as accommodative intraocular lens (A-IOL) implants, refractive lens exchange (RLE), scleral muscle band implants for axial length modulation, multi-index liquid filled lenses for near/far multifocality, monovision, presby-LASIK for corneal asphericity, electro-active lenses and capsular replace/refill procedures with customized gels, and the like. Generally, these procedures are directed at producing improved near focus, however, these techniques are invasive and/or are characterized by undesirable side effects. LASIK for example also alters the distance acuity, contrast sensitivity and wavefront, which may or may not be desired. Other common presbyopia therapies/methods include multifocal/diffractive/progressive spectacles and contact lenses, as well as Ortho-K and physiological eye exercise based vision improvements.

Generally, scleral mechanism claims are based on non-Helmholtzian theories (and thus are contrary to the overwhelming scientific body of evidence). SEBs have rarely shown objective and stable gains in accommodative amplitude measurements. Additionally, the SEB surgery itself is typically a highly invasive complicated and lengthy process with some reported implant slippage. LaserAce/VisioDynamic surgery involves multiple deep scleral perforations and a biological patch per quadrant for improved wound healing, again making for greater lengthier and costlier steps. ACS/LRI works by post-equatorial scleral weakening, which presents stability concerns, and the effectiveness of these treatments is not well established. Intraocular optics implants(IOLs/Inlays/Onlays etc) operate statically and so do not necessarily improve accommodative amplitude(AA) but improve depth of focus or trade off contrast if Multi Focal or Diffractive. Also, A-IOLs/RLEs are invasive procedures (typically useful at the cataractous stage), primarily assuming normal geometric movement/alignment of the ciliary/equatorial complex, but not corrective of it and are sometimes known to result in visual contrast loss. If the patient is not nearing the cataracterous age, the patient may well consider delaying treatment due to these concerns. Customized gel capsular refilling has shown initial progress, but is currently still very much a lab procedure. MV is generally a compromise treatment (near vs far focus tradeoff) requiring lengthy adaptation, thereby producing suboptimal results and no real AA gains. Scleral muscle prosthesis/implants/bands/rings, while dynamic in modulating axial length, are controlled manually and require external power and are not able to restore natural physiological accommodation, and furthermore, successful clinical results have not been confirmed. Multi-refractive index/weight liquid lenses offer implants another unique approach with potentially even bi-focality, but are very much at an early stage of prototyping.

Therefore, there is a need in the art for a method and apparatus for treating presbyopia using a noninvasive or minimally invasive treatment process addressing the previously described limitations of other presbyopia treatment methods.

### SUMMARY OF THE INVENTION

According to the present invention, excitation and radiation techniques and apparatus for performing customized modulation, particularly elasto-modulation, of ocular tissue in an annular region around the cornea, such as for the purpose of ciliary translocation (ACT) or promotion of aqueous outflow to relieve intra-ocular pressure are provided utilizing a trans-conjunctival incision-less scleral treatment. Laser radiation is for example applied to scleral tissue to decrease local rigidity, acqueous outflow blockage, zonular slack, and/or alter equatorial offsets between lens and ciliary muscles as a treatment for presbyopia by way of example.

According to an embodiment of the invention, a method of producing anterior and radial ciliary translocation in a living eye is provided wherein the eye is irradiated at a plurality of predetermined locations (serially or simultaneously) posterior to the limbus of the eye such that the limbus is not significantly heated or damaged, also determining a radial endpoint associated with the intended ciliary translocation and bounding the region of irradiation near the endpoint, and then terminating the irradiating process within the boundaries of the endpoint after an effective level of radiation has been absorbed in the target region between the limbus and the endpoint. The length of time and intensity of irradiation of the target region is chosen to attempt to optimize the shrink rate and the post-shrink stability of the scleral region.

According to another embodiment of the present invention, an apparatus for producing ciliary translocation in an eye is provided. The apparatus includes a radiation source adapted to irradiate the eye at a plurality of predetermined locations posterior to the limbus of the eye. The apparatus also includes a processor adapted to determine an endpoint associated with ciliary translocation and terminate the irradiating process.

Numerous benefits are achieved using the present invention over conventional techniques. Some embodiments provide treatment for conditions of the eye, including presbyopia, lenticular aberrations, and glaucoma utilizing a noninvasive procedure. Additionally, embodiments of the present invention provide for non-invasive or minimally invasive treatments with reduced side effects compared to conventional techniques. Treatments provided according to embodiments of the present invention described herein may result in patients not needing prosthetic devices, such as spectacles. Depending upon the embodiment, one or more of these benefits may exist. These and other benefits have been described throughout the present specification and more particularly below.

Various additional objects, features and advantages of the present invention can be more fully appreciated with reference to the detailed description and accompanying drawings that follow.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a simplified illustration demonstrating the Helmholtz theory of accommodation;

FIG. 2 is a simplified illustration demonstrating the Schachar theory of accommodation;

FIGS. 3A, 3B, and 3C are simplified illustrations of the anterior segment of the eye, the flow of aqueous humor in the eye, and the ciliary body, respectively;

FIG. 4A is a simplified schematic diagram illustrating the placement of laser energy on the conjunctiva;

FIGS. 4B and 4C are simplified schematic diagrams illustrating preoperative and postoperative ocular conditions according to an embodiment of the present invention;

FIG. 4D is a simplified schematic diagram illustrating postoperative ocular conditions according to an alternative embodiment of the present invention;

FIG. 5 is a bar graph illustrating preoperative and postoperative uncorrected near visual acuity for ten patients;

FIG. 6 is a simplified schematic illustration of components of the eye;

FIGS. 7A and 7B are simplified illustrations of the flow of aqueous humor for various stages of glaucoma;

FIG. 8 is a simplified flowchart illustrating a method of performing anterior ciliary translocation according to an embodiment of the present invention;

FIG. 9 is an illustration of a porcine eye with a temperature probe inserted;

FIG. 10 is a graph illustrating temperature distributions achieved using an embodiment of the present invention; and

FIG. 11 is an illustration of an applicator according to an embodiment of the present invention.

### DETAILED DESCRIPTION OF SPECIFIC EMBODIMENTS

According to the present invention, biomedical techniques are provided for modulation of tissue, that is manipulation of the tissue. More particularly, the invention includes a method and apparatus for performing customized anterior ciliary translocation. Merely by way of example, the invention has been applied to using the delivery of laser radiation to scleral tissue to decrease local rigidity, acqueous outflow blockage, zonular slack, and/or alter equatorial offsets between lens and ciliary muscles as a treatment for presbyopia by way of example. But it would be recognized that the invention has a much broader range of applicability.

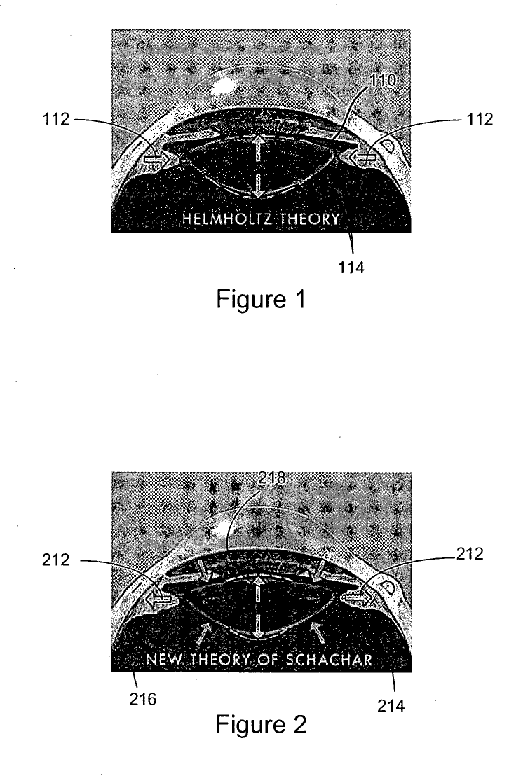

FIG. 1 is a simplified illustration demonstrating the Helmholtz theory of accommodation. According to the Helmholtz theory of accommodation, the ciliary muscles relax for near vision and allow the anterior surface of the lens to become more convex. Therefore, when an individual focuses his or her eye for near vision, the ciliary muscles in the eye relax as illustrated by arrows 112 in FIG. 1 and release tension on the lens ligaments, allowing the lens to thicken. The thickening of the lens 110 is illustrated by arrows 114 in FIG. 1.

FIG. 2 is a simplified illustration demonstrating the Schachar theory of accommodation. According to the theory of Schachar, the ciliary body contracts externally in accommodation. This contraction, illustrated by arrows 212 in FIG. 2, causes the equatorial zonules to stretch, thereby actively steepening the central lens surface and allowing accommodation. As illustrated in FIG. 2, the equatorial fibers of the ciliary muscles pull directly on the ends of the lens as illustrated by arrows 214. The tensile forces exerted on the lens result in a flattening of the periphery of the lens, illustrated by arrows 216 and an increase in thickness of the lens as illustrated by arrows 218 and the dashed curves on the anterior and posterior surfaces of the lens.

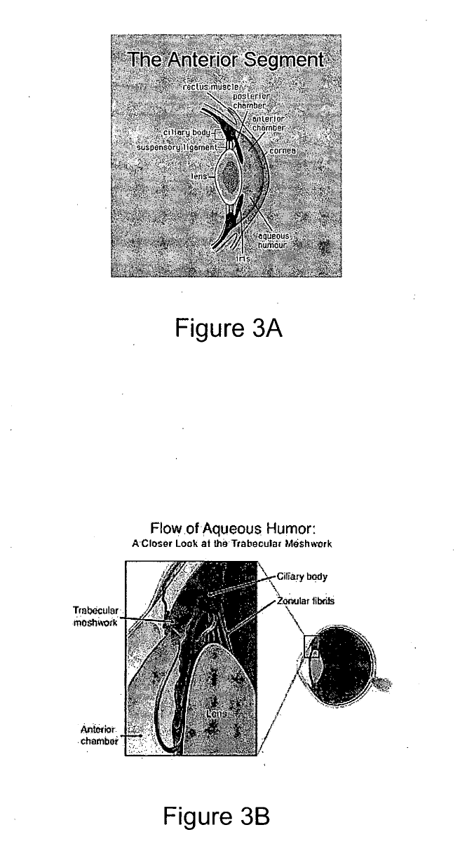

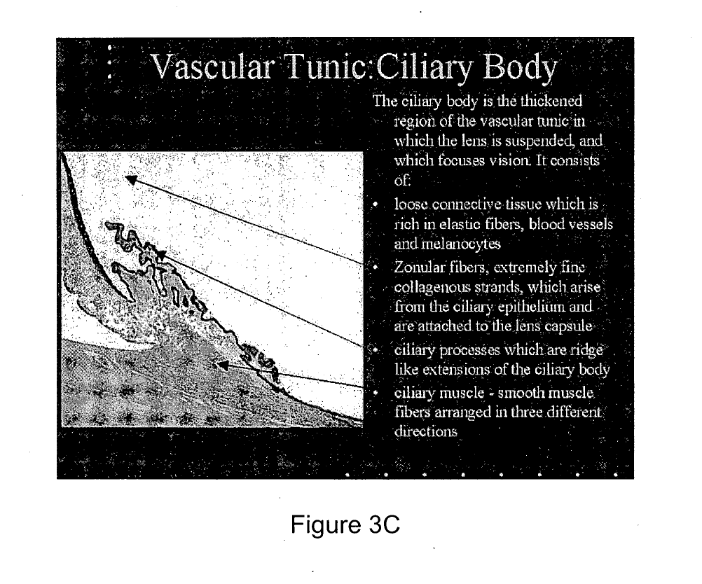

FIGS. 3A, 3B, and 3C are simplified illustrations of the anterior segment of the eye, the flow of aqueous humor in the eye, and the ciliary body, respectively.

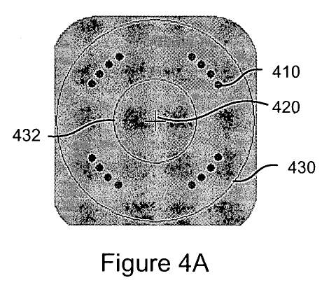

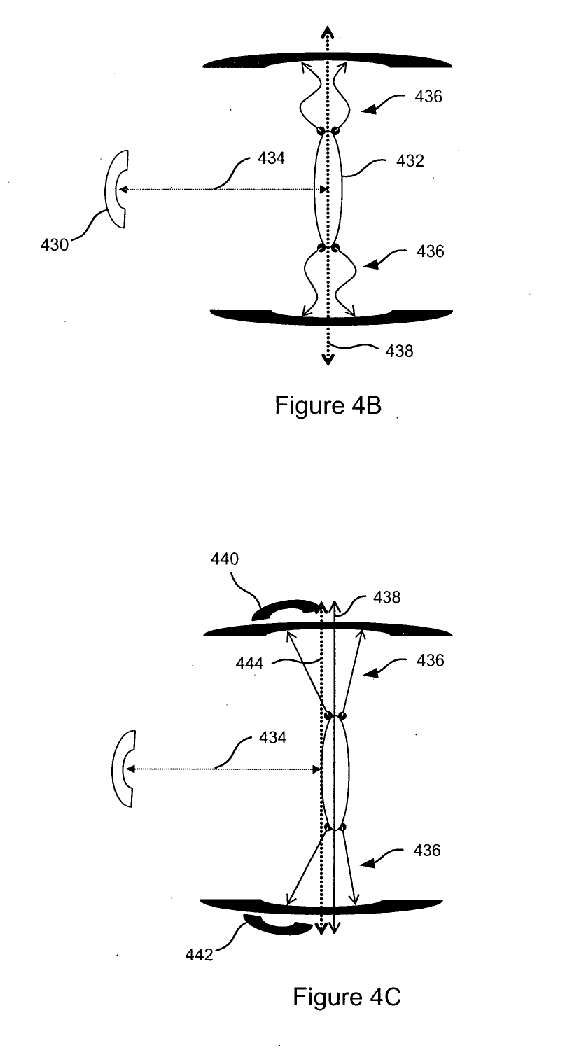

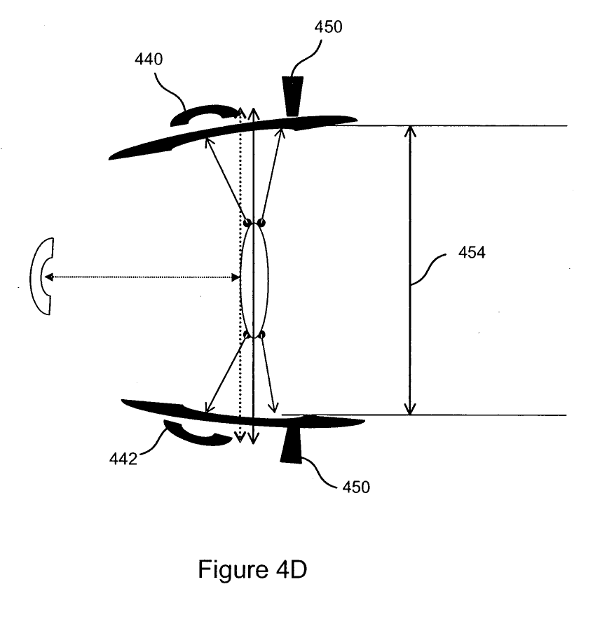

FIG. 4A is a simplified schematic diagram illustrating the placement of laser energy on the conjunctiva. According to the present invention, methods and systems are provided to perform anterior ciliary translocation (ACT) utilizing a transconjunctival incisionless scleral treatment to treat presbyopia, lenticular aberrations, glaucoma, and other medical conditions. In FIG. 4A, the treatment region is illustrated by circle 430 and the lens is illustrated by circle 432.

As illustrated in FIG. 4A, a predetermined target treatment region is outlined and/or a number of target spot marks 410 per quadrant are made on the conjunctiva. In the embodiment illustrated in FIG. 4A, the spot marks are placed at preselected locations associated with the lens equator. In a particular embodiment, five spot marks per arc in each quadrant are made at radial locations ranging from 0.5 mm to 4 mm behind the limbus. Generally, the cornea has a diameter of approximately 10 mm to 11 mm. Therefore, embodiments of the present invention which treat the conjunctiva near the lens equator do not deliver laser radiation to the cornea. In other embodiments, the number of spot marks per arc in each quadrant are increased, for example, to seven or more, or decreased as appropriate to the particular applications.

Measurement of the mechanical properties of the sclera are made in some embodiments using segmented linear arrays of ultrasound transducers. Accordingly, positional location of the ciliary body with respect to the cornea are provided herein. The transducers are arranged in arcs centered on the center of the eye. Merely by way of example, the transducers may be arranged to overlap the angular placement of spots illustrated in FIG. 4A.

The angular placement of the spots in an embodiment similar to that illustrated in FIG. 4A, but with five spots per quadrant, is typically performed according to the angles provided in Table 1. In FIG. 4A, the angles are measured with respect to a vertical line passing through the cross-hairs 420 at the center of the eye.

TABLE 1

Quadrant

| Angle (°) | | I | II | III | IV |
|---|---|---|---|---|---|
| Spot | #1 | 33 | 123 | 213 | 303 |
| | #2 | 39 | 129 | 219 | 309 |
| | #3 | 45 | 135 | 225 | 315 |
| | #4 | 51 | 141 | 231 | 321 |
| | #5 | 57 | 147 | 237 | 327 |

As will be evident to one of skill in the art, the selection of the number of spots and the angular placement of the spots may be selected to optimize delivery of laser power. As a result, in some embodiments, the number of spots per quadrant will be greater than or less than equal to five; the angular placement of the spots will be centered at angles other than 45°; and the radial distance from the center of the eye to the spots will vary. In the embodiment illustrated in FIG. 4A, the placement and spacing of the laser spots in groupings is selected to preserve veins and blood flow in the eye as well as the muscles associated with extraocular movement (EOM). As will be described in additional detail below, thermal energy associated with laser pulses is directed to predetermined portions of the eye in predetermined fluences.

In a specific embodiment of the present invention, a Ho:YAG laser operating at 2.1 μm is utilized to deliver five sequential spots per quadrant, wherein each spot comprises 15 pulses having 184 mJ/pulse. For a laser system operating at 5 Hz, a treatment time of 15 seconds per quadrant (3 seconds per spot) will provide the desired 15 pulses per spot. For a single arc of 20 spots as illustrated in FIG. 4A, a total treatment time of 60 seconds is utilized by the specific embodiment discussed above. In other embodiments, other CW or Pulsed, contact or non-contact sources adapted to provide for shrinkage of ocular tissue are utilized, including other infrared sources, microwave sources, UVA sources, ultrasound sources, electro-resistive sources, Peltier sources, temperature controlled specialized fluid baths and the like.

Moreover, in some embodiments, the number of arcs at which laser spots are delivered is increased to increase the laser fluence. In a particular embodiment, two or more arcs are provided at similar or differing angles, differing from the spots illustrated in FIG. 4A by the radial distance from the center of the eye to these additional arcs. As will be evident to one of skill in the art, the combination of differing numbers of spots, spot angle, laser energy fluence per pulse, arcs, pulse length, and the like may be selected to provide substantially equivalent results or outcomes. Of course, the combination of differing numbers of these variables may also be selected to increase or decrease the effects produced by embodiments of the present invention.

FIG. 4B is a simplified schematic diagram illustrating preoperative ocular conditions according to an embodiment of the present invention. As illustrated in FIG. 4B, the cornea 430 is illustrated as separated from the lens 432 by a distance 434. In some embodiments, the distance 434 is referred to as an anterior chamber depth (ACD). The line 438 is aligned with the center of the lens 432. Zonular slack, illustrated by waviness in ciliary structures 436 is present in the preoperative condition illustrated in FIG. 4B.

Some estimates of lens growth place lens growth at 20 μm/year, which means to reverse 10 years of zonular slack lenticular growth, about 200 μm of actual shrinkage (equivalent to 600 μm for volume shrinkage at a 1/3 ratio) is an appropriate treatment for most patients. Ray tracing depicts approximately 100 μm of anterior movement of the natural lens to be correlated with about one diopter of accommodative amplitude for a normal eye. MRI studies that have been reported show normal ciliary function and lenticular elasticity especially in early presbyopes but a lens-ciliary complex crowding, scieral rigidity and anterior growth of the lens along with the ciliary muscle apex.

FIG. 4C is a simplified schematic diagram illustrating postoperative ocular conditions according to an embodiment of the present invention. As illustrated in FIG. 4C, laser radiation, for example from a Ho:YAG laser, is directed at treatment regions 440 and 442. Heat shrinkage resulting from a local increase in eye temperature due to absorption of the laser radiation induces translocation of the ciliary muscles in the anterior direction. Accordingly, dynamic anterior lens movement is made possible during accommodation. Additionally, reductions in zonular slack, illustrated by the straightening of ciliary structures 436, improve the efficiency of force coupling with the capsular bag.

As illustrated in FIG. 4C, the center of the lens 438 has been translocated in the horizontal direction from the position illustrated by line 444 to the position shown in FIG. 4C. The translocation of the lens resulting from treatments provided according to embodiments of the present invention may differ depending on the particular treatment modality employed. As an example, translocation in other directions may be performed. Moreover, by adjusting the amount of slack removed from the ciliary structures, correction of lenticular aberrations may be made. Furthermore, some embodiments maintain the position of the lens in the same horizontal plane illustrated by line 438 in FIG. 4B.

According to embodiments of the present invention, heat shrinkage of transconjunctival tissue near the scleral spur is induced. In a particular embodiment, a temperature of approximately 75° C. is created in regions of the eye associated with the laser radiation for approximately 4 seconds. The associated heat shrinkage of the ocular tissues provides anterior translocation of the scleral spur and a net accommodative amplitude increase of between one and three diopters for every hundred microns of decrease in zonular slack in some embodiments. Embodiments of the present invention reduce the slack present in the ciliary structures, tightening the capsular coupling and increasing the effectiveness of lenticular reshaping. As described throughout the specification, some embodiments of the present invention utilize a noninvasive (no cutting of the ocular tissue) technique employing topical anesthetic that is corneal and limbal. Some embodiments of the present invention are titrated to anterior chamber depth (ACD) or reduce or customize optical aberrations during the treatment process.

Embodiments of the present invention violate neither the Helmholtz nor Schachar conceptual theories, but provide treatment options consistent with both theories. Ray tracing has shown that dynamic anterior motion of the lens during accommodative effort serves to provide improvements in vision. In addition to presbyopia, embodiments of the present invention provide methods and apparatus adapted to treat lenticular aberrations as well as glaucoma, discussed in more detail below.

Intraocular pressure (IOP) is generally not increased by treatments performed utilizing embodiments of the present invention. In fact, as described below, the IOP may be reduced in glaucoma patients. Additionally, based on our experiments, the manifest refractive spherical equivalent (MRSE) is unaffected, scleral shrinkage is under 1 mm of depth and about 1 mm of width per spot utilizing the four arcs illustrated in FIG. 4A.

In some embodiments of the present invention, a scleral stiffening of certain regions adjacent to the treated area using the heat shrinkage techniques provided herein is performed postoperatively to the ensure longevity of the treatment. For example, scleral stiffening via UVA/riboflavin may be performed. Additional details regarding scleral stiffening via UVA/riboflavin treatment are provided in commonly assigned U.S. patent application Ser. No. 10/958,711, filed Oct. 4, 2004 and incorporated by reference for all purposes. Scleral hardening around the treatment region will serve to increase the useful lifetime of the treatment provided by embodiments of the present invention for some patients.

In alternative embodiments, a small region is ablated and sutured anterior to the equator to yield a similar biomechanical effect but with the additional step of a quad arc ablative/femtosecond laser type treatment. Generally, minor postoperative pain usually lasting one to several hours immediately following the procedure is easily managed by physician and/or the patient. Experiments have shown that real time (RT)-intraoperative WF guided ACT has achieved improved outcomes compared to conventional techniques. In some studies, desired results have been achieved immediately following the ACT procedure.

As illustrated by distances 434 in FIGS. 4B and 4C, the position of the equatorial plane with respect to the cornea is unchanged when the eye is in the unaccommodated state. Thus, the ACD is reduced only during accommodation. In some embodiments, the amplitude of accommodation (AA) is approximately one diopter for every 100 μm of decrease in zonular slack. Generally, the greater the translocation and the pliancy of the ciliary tissues, the greater the AA. As will be evident to one of skill in the art, a similar trend is appropriate for circumferential expansion. In some embodiments, anterior vector forces are present during treatment. Moreover, lenticular diameter expansion is utilized in some embodiments to reduce higher order aberrations present in the eye of the patient. Furthermore the posterior sclera exhibits increased postoperative pliancy, which enhances transmission fluid forces in vitreous members.

FIG. 4D is a simplified schematic diagram illustrating postoperative ocular conditions according to an alternative embodiment of the present invention. As illustrated in FIG. 4D, a combination of thermal shrinkage, illustrated by treatment regions 440 and 442, and a radial keratotomy (RK)/laser incision treatment is utilized according to an embodiment of the present invention. The RK/laser incision treatment is illustrated by incision 450 in FIG. 4D. In some embodiments, incision 450 on the posterior sclera will expand the diameter 454 and result in the vitreous yielding to anterior movement of the equatorial plane. In some embodiments, this combination treatment will result in increased pliancy of the vitreous. Additionally, reduced ciliary slack due to posterior scleral diameter expansion will result in improved efficiency of force coupling with the capsular bag. As illustrated by distance 454, the diameter of the posterior sclera is increased with respect to the diameter illustrated in FIG. 4B. As discussed in relation to FIG. 4C, in some embodiments, scleral stiffening of the region treated using the combination heat shrinkage and RK/laser incision techniques provided herein is performed postoperatively to the ensure longevity of the treatment.

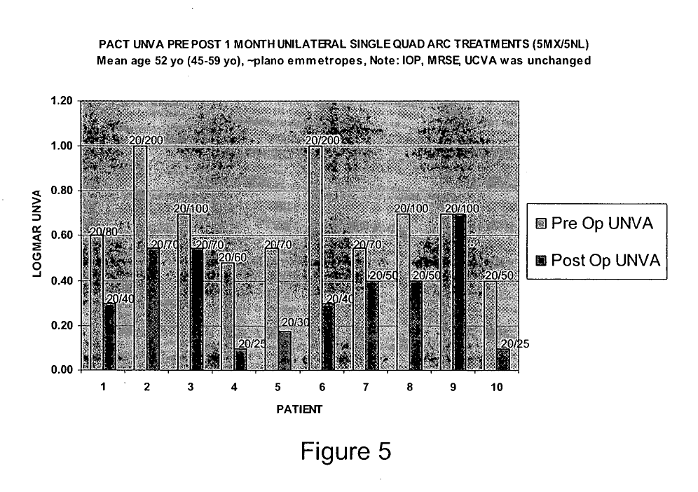

FIG. 5 is a bar graph illustrating preoperative and postoperative uncorrected nearer visual acuity (UNVA) for ten patients. As illustrated in FIG. 5, the logarithm of the minimum angle of resolution (LogMar) for UNVA is plotted for each of ten patients before and after treatment utilizing methods and apparatus provided according to embodiments of the present invention. For example, for patient number one, preoperative UNVA was 20/80 whereas postoperative UNVA was 20/40. Improvements in UNVA were observed for nine of the 10 patients in the study. An item of note is that five of the patients received treatments in Mexico, while five of the patients received treatments in the Netherlands.

The mean age of the patients for which experimental results are illustrated in FIG. 5 was 52 years old, with a range of ages from 45 to 59 years of age. For these 10 patients, IOP, MRSE, and uncorrected visual acuity (UCVA) was unchanged.

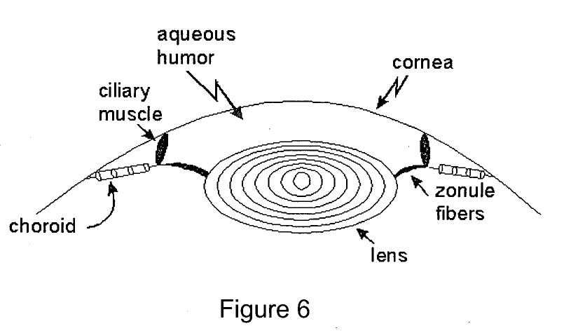

FIG. 6 is a simplified schematic illustration of components of the eye. As illustrated in FIG. 6, when the ciliary muscle 610 is relaxed, the choroid 612 acts like a spring pulling on the lens 614 via the zonule fibers 616. As a result of this process, the flatness of the lens is increased. When the ciliary muscle contracts, the choroid 612 is stretched, releasing the tension on the lens. As result of this process, the thickness of the lens is increased.

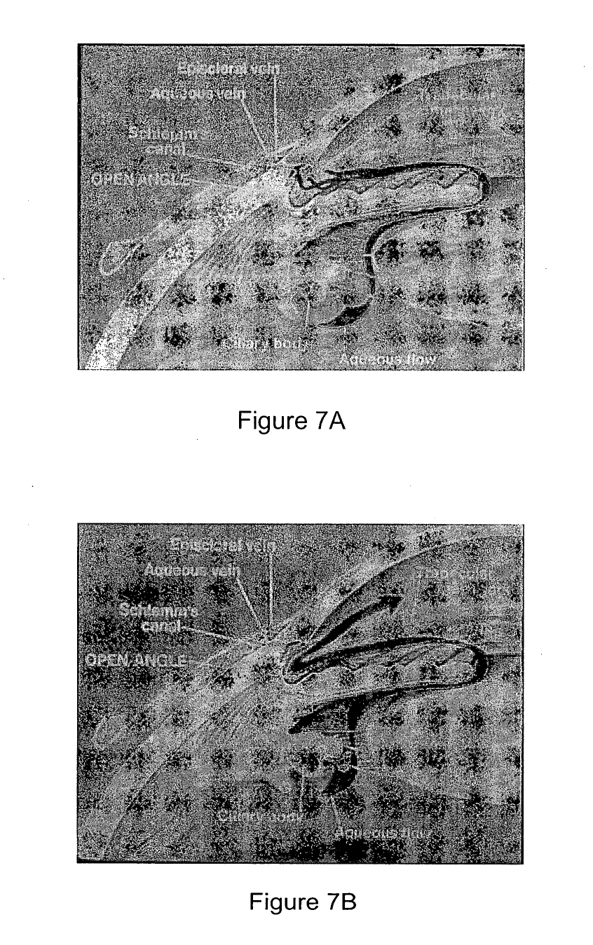

FIGS. 7A and 7B are simplified illustrations of the flow of aqueous humor for various stages of glaucoma. As illustrated in FIG. 7A, the aqueous flow through the open angle is decreased. This condition is generally referred to as open angle glaucoma. Utilizing embodiments of the present invention, thermal treatment of the ciliary body results in an increase in the aqueous flow through the open angle as illustrated in FIG. 7B. In some situations, embodiments of the present invention provide an alternative treatment to endoscopic cyclophotocoagulation (ECP).

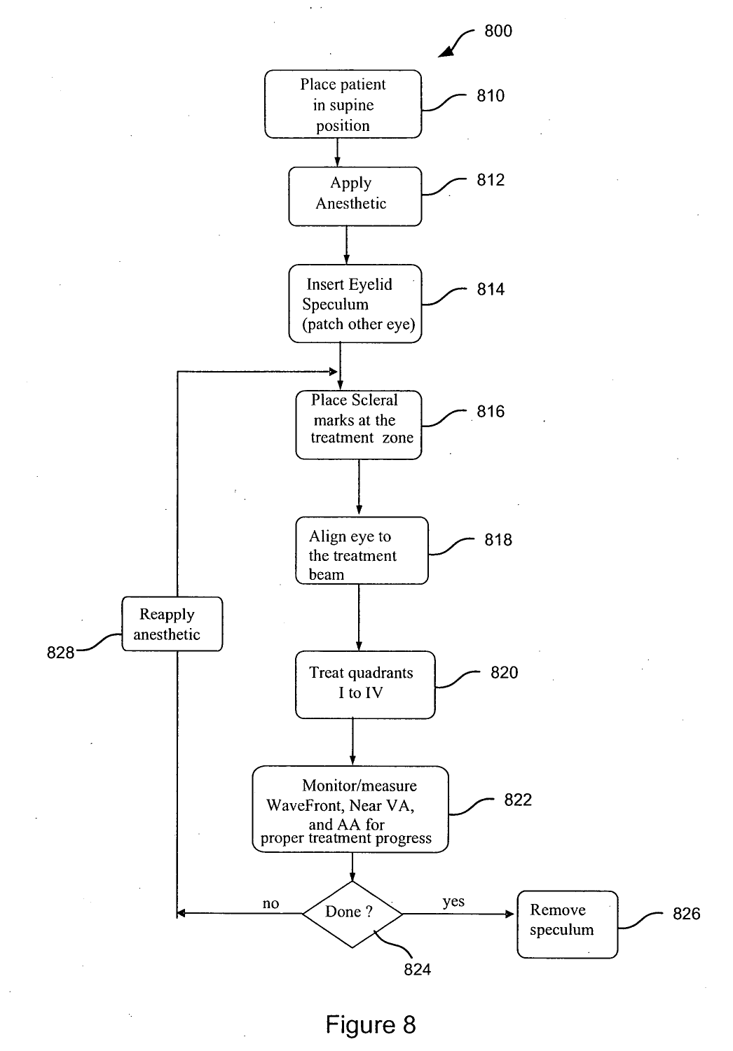

FIG. 8 is a simplified flowchart illustrating a method 800 of performing anterior ciliary translocation according to an embodiment of the present invention. In step 810, the patient is placed in the supine position and an anesthetic is applied to the patient's eye or eyes depending on the particular procedure. An eyelid speculum is inserted in the patient's eye(s) in step 814. In some procedures, a patch is applied to a non-treated eye. Scleral marks are placed on the treatment zone in step 816. Although the embodiment illustrated in FIG. 8 utilizes marks, this is not required by the present invention. In alternative embodiments, laser system software is utilized that registers the delivery of the laser pulses to the eye, the speculum, or other structure. One of ordinary skill in the art would recognize many variations, modifications, and alternatives.

An alignment step is performed in step 818 to align the eye to the treatment beam. In other embodiments, the treatment beam is aligned to the patient's eye. The treatment is performed in step 820. Generally, the treatment proceeds from one quadrant to the next, sequentially delivering laser pulses to the various marked treatment locations. In alternative embodiments, a portion of the laser pulses for a given quadrant are delivered, pulses are delivered to another quadrant, and the laser pulses are subsequently delivered to the original quadrant. One of ordinary skill in the art would recognize many variations, modifications, and alternatives.

In step 822, monitoring of the treatment process and measurement of the results are performed, including monitoring/measuring of the laser wavefront, near visual acuity, and AA for ensuring proper progress of the treatment process. In some embodiments, step 822 is performed simultaneously with step 820, whereas in other embodiments, these steps are performed in combinations of sequential and simultaneous manners. In step 824, a determination is made of whether or not the treatment process is completed.

If the process is completed as desired, the speculum is removed in step 826 and the treatment process is stopped. In, on the other hand, additional treatment is warranted, the anesthetic is reapplied in step 828 and the treatment process is reinitiated at step 816.

It should be appreciated that the specific steps illustrated in FIG. 8 provide a particular process flow according to one embodiment of the present invention. Other sequences of steps may also be performed according to alternative embodiments. For example, alternative embodiments of the present invention may perform the treatment steps outlined above in a different order. Moreover, the individual steps illustrated by this figure may include multiple sub-steps that may be performed in various sequences as appropriate to the individual step. For example, the application of anesthetic or the placement of scleral marks at the treatment zone may include multiple steps that may be defined in various sequences within the scope of the present invention. Furthermore, additional treatment steps may be added or removed depending on the particular applications. One of ordinary skill in the art would recognize many variations, modifications, and alternatives.

Other embodiments of the present invention include the use of high-intensity ultrasound energy as a modality for controlled modification of ocular tissue. A specific application of this technology includes use for precise, localized and symmetric heating of portions of the sclera, ciliary muscle, and/or zonules as a means of improving accommodation, correcting optical aberrations or errors, and generating optical correction. Thus, we have conducted experiments with prototype ultrasound heating applicators on porcine eyes.

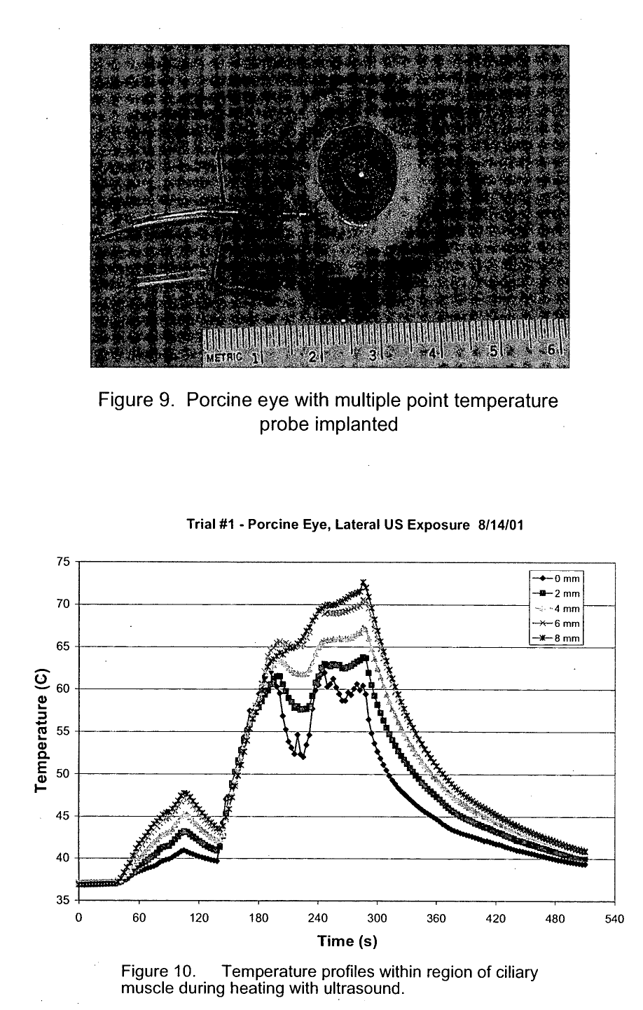

The first set of experiments were designed to evaluate Ultrasound-Induced Temperature Distributions (UITD) and the ultrasound penetration characteristics of the eye to determine if ultrasound energy in the 4-10 MHz range could be selectively delivered to the ciliary muscle and surrounding structures without exposing the lens to thermal energy. In an experiment, porcine eyes were placed within a 37° C. water bath and allowed to come to equilibrium. Miniature temperature probes which consisted of multiple thermocouple sensors within an 18 g stainless steel needle were placed adjacent to ciliary muscle beneath the sclera (˜1-3 mm deep). A small ultrasound transducer (2 cm diameter disk) operating at 4 MHz was placed within the immersion bath and directed at the temperature sensor. The porcine eye with the temperature probe inserted is shown in FIG. 9.

The ultrasound energy was applied and the temperature distributions were measured, as shown in FIG. 10. It is generally accepted that temperatures greater than 50° C. are required to coagulate and shrink structural collagen. These results demonstrate that ultrasound can penetrate the sclera and generate high temperatures within selected regions around the ciliary muscles. In alternative designs, we anticipate devices that can selectively heat portions of the eye for 1-5 minutes at temperatures high enough to induce modification.

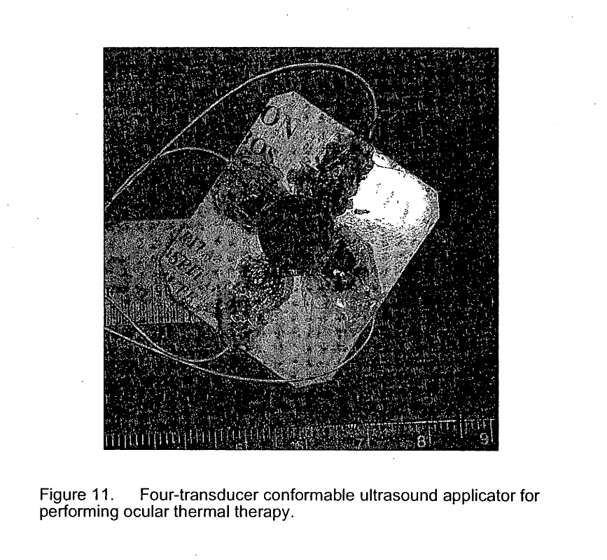

In a second set of experiments, a prototype ultrasound applicator was designed specifically for testing the concept of symmetrical heating around, but not including, the lens. Four small rectangular transducer segments (3 mm×4 mm), operating ˜7 MHz, were mounted in a symmetrical circular pattern at 18 mm diameter, separated by 90°. The mounting substrate was a semi-flexible plastic and was designed with a central hole for alignment on the eye and allowing for future optical measurement of changes in lens distortion. A picture of the applicator is shown in FIG. 11. Similar to the above setup, porcine eyes were placed within a 37° C. water bath. A red dye was injected within the lens to allow visualization of distortion, physical movement, or change of shape within the lens. The ultrasound energy was delivered at 10-15 W per transducer segment for 1-5 minutes. The eye/lens was photographed with a digital camera immediately before and after heating to visualize any thermally induced changes within the eye.

It was determined that the use of before/after images for determining changes to the lens was not as accurate or reliable as desired. Improvements in design and fabrication techniques can easily overcome issues related to power delivery and applicator robustness and achieve selective heating. The use of non-invasive optical measurements of the lens will enable the evaluation of the feasibility of this technology.

The inventors are aware that high-intensity focused ultrasound technology is being utilized to treat ocular cancers in a few institutions, such as Dr. Lizzi's work in Riverside Clinic, NY. These devices use tightly focused beams to direct the heating to the posterior wall of the eye, including the retina and regions around the fovea. The precise positioning of the focus is achieved using A-mode ultrasound scans. Embodiments of the present invention apply heat to small discrete regions in the anterior eye, just below the sclera. From our work and others, we know that ultrasound energy can penetrate the sclera and localize heat to deeper portions of the eye, demonstrating the basic feasibility of localizing heating to discrete portions of the eye. Accordingly, we are developing ultrasound heating technology specific to controlled modification of the ciliary muscles and zonules, along with the implementation of new measurement methods for verification and control of the treatment.

Future experiments will be used to:
1. Determine whether flat, curved, or lightly focused transducers provide the best heating patterns.
2. Optimize the ultrasound frequency to minimize heating of the sclera, but limit tissue modification to depths of 3-5 mm.
3. Implement active applicator surface cooling as a means of protecting the sciera and increasing the maximum temperature within the target region.
4. Design a conformable applicator interface for proper placement and alignment on the eye.
5. Perform a large series of acoustic measurements and thermal dosimetry measurements in eyes to support above design studies.
6. Integrate optical measurements of lens distortion as a means of evaluating the efficacy of treatments provided according to embodiments of the present invention.

It is also understood that the examples and embodiments described herein are for illustrative purposes only and that various modifications or changes in light thereof will be suggested to persons skilled in the art and are to be included within the spirit and purview of this application and scope of the appended claims in connection with for example a much broader range of applicability related to the anterior ciliary translocation in an eye.

What is claimed is:

1. A method for therapeutically manipulating regions of a living human eye having an ocular lens, a cornea, a sclera, a ciliary muscle, a ciliary body complex, a trabecular meshwork, and zonular ligaments, the method comprising:
exciting a volume of the eye limited to a bounded annular targeted region posterior to the cornea with a sufficient dosage of energy to modulate the targeted region to permit an effective physical change within ocular tissue of the targeted region and immediately adjacent regions; and
terminating the exciting step prior to onset of irreversible damage to the ocular lens and the cornea.

2. The method of claim 1 wherein the exciting step elasto-modulates the trabecular meshwork to effect aqueous outflow and thereby reduce ocular pressure.

3. The method of claim 1 wherein the exciting step elasto-modulates the sclera around the ciliary body complex thereby mitigating presbyopia.

4. The method of claim 1 wherein the exciting step is effected by electromagnetic energy radiation in the spectrum including one of radio frequency, microwave frequency, infrared frequency, visible light frequency, and ultraviolet frequency.

5. The method of claim 1 wherein the exciting step is effected by ultrasonic energy coupled to the annular targeted region.

6. The method of claim 1 wherein the exciting step is effected by direct mechanical vibrating applied to the annular targeted region.

7. The method of claim 1 wherein the exciting step is effected by direct mechanical abrasion of the surface of the annular targeted region.

8. The method of claim 1 wherein the exciting step is effected by femto-second pulsed infrared laser irradiation sufficient to ablate below the surface of the annular targeted region without ablation of the lens.

9. The method of claim 1 further including the step of monitoring at least one parameter of the ocular mechanism of the human eye as indicia of the terminating of the exciting step.

10. The method of claim 9 wherein the at least one parameter is at least one of near visual acuity and amplitude of accommodation.

11. The method of claim 9 wherein the monitoring step includes measuring intraocular pressure.

12. The method of claim 9 wherein the monitoring step includes measuring lens thickness.

13. The method of claim 9 wherein the monitoring step includes measuring the boundary between the lens and the ciliary complex.

14. The method of claim 1 further comprising providing a plurality of scleral registration marks representing a plurality of predetermined locations at the outer boundary of the bounded annular targeted region.

15. The method of claim 14 wherein a first number of the plurality of scleral registration marks is arranged in a first arc located at a first distance from a center point of the eye.

16. The method of claim 15 wherein the first number of the plurality of scleral registration marks is located in a first quadrant.

17. The method of claim 14 wherein a second number of the plurality of scleral registration marks is arranged in a second arc located at a second distance from the center point of the eye, the second arc being located in a second quadrant.

18. The method of claim 17 wherein the first distance and the second distance are substantially equal.

19. The method of claim 1 wherein the predetermined locations posterior to the cornea are along an equatorial region of the eye.

20. The apparatus of claim 1 wherein the exciting step comprises a thermo-coagulating laser.

21. The method of claim 19 wherein the exciting step comprises pulsing a laser to distribute a plurality of laser pulses along an arc centered at a center point of the eye.

22. An apparatus for therapeutically manipulating regions of a living human eye having an ocular lens, a cornea, a sclera, a ciliary muscle, a ciliary body complex, a trabecular meshwork, and zonular ligaments, the apparatus comprising:
an energy source operative to excite a volume of the eye limited to a bounded annular targeted region posterior to the cornea; and
a processor controlling the energy source and operative to:
1) control application of a sufficient dosage of energy to modulate the targeted region to permit an effective physical change within ocular tissue of the targeted region and immediately adjacent regions; and to
2) terminate the exciting step prior to onset of irreversible damage to the ocular lens and the cornea.

23. The apparatus of claim 22 wherein the exciting source comprises a source of electromagnetic energy radiation in the spectrum including one of radio frequency, microwave frequency, infrared frequency, visible light frequency, and ultraviolet frequency.

24. The apparatus of claim 22 wherein the exciting source comprises an ultrasonic energy generator coupled to the annular targeted region.

25. The apparatus of claim 22 wherein the exciting source comprises a direct mechanical vibrator adapted to be applied to the annular targeted region.

26. The apparatus of claim 22 wherein the exciting source comprises a direct mechanical abrader operative on the surface of the annular targeted region.

27. The apparatus of claim 22 further comprising a detector operative to monitor at least one parameter associated with the eye.

28. The apparatus of claim 27 wherein the at least one parameter is at least one of near visual acuity and amplitude of accommodation.

29. The apparatus of claim 22 further comprising a marker operative to provide a plurality of scleral registration marks representing the plurality of predetermined locations.

30. The apparatus of claim 29 wherein a first plurality of the plurality of scleral registration marks is arranged in a first arc located at a first distance from a center point of the eye.

31. The apparatus of claim 30 wherein the first plurality of the plurality of scleral registration marks is located in a first quadrant.

32. The apparatus of claim 29 wherein a second plurality of the plurality of scleral registration marks is arranged in a second arc located at a second distance from the center point of the eye, the second arc being located in a second quadrant.

33. The apparatus of claim 32 wherein the first distance and the second distance are substantially equal.

34. The apparatus of claim 22 wherein the predetermined locations posterior to the limbus of the eye are located at an equatorial region of the eye.

35. The apparatus of claim 22 wherein said irradiating of the eye comprises delivering a plurality of laser pulses to the eye.

36. The apparatus of claim 35 wherein the radiation source comprises a Ho:YAG laser.

37. The apparatus of claim 35 wherein the plurality of laser pulses is distributed along an arc centered at a center point of the eye.
